(19) United States
(12) Patent Application Publication
(10) Pub. No.: US 2014/0276227 A1
(43) Pub. Date: Sep. 18, 2014

(54) SLEEP MANAGEMENT IMPLEMENTING A WEARABLE DATA-CAPABLE DEVICE FOR SNORING-RELATED CONDITIONS AND OTHER SLEEP DISTURBANCES

(71) Applicant: Gerardo Barroeta Pérez, San Francisco, CA (US)

(72) Inventor: Gerardo Barroeta Pérez, San Francisco, CA (US)

(73) Assignee: AliphCom, San Francisco, CA (US)

(21) Appl. No.: 13/830,927

(22) Filed: Mar. 14, 2013

(51) Int. Cl. A61B 7/00; A61B 7/04; A61B 5/00

(52) U.S. Cl. CPC A61B 7/003; A61B 5/7455; A61B 5/746; A61B 5/4818; A61B 5/7282; A61B 7/04; A61B 5/0002; A61B 5/6803; A61B 5/681; A61B 5/6898
USPC 600/586

(57) ABSTRACT

Embodiments relate generally to electrical and electronic hardware, computer software, wired and wireless network communications, and wearable computing devices for sensing health and wellness-related physiological characteristics. More specifically, an apparatus and method can provide for snore detection and management implementing either wearable devices or non-wearable devices, or a combination thereof. In some examples, a method includes receiving an acoustic signal, characterizing the acoustic signal as a snoring sound to determine presence of a snoring condition, and transmitting a notification signal to cause notification of the detection of the snoring sound. Optionally, the method can include receiving the notification signal, and causing a notification source to notify of the presence of a snoring condition or any other sleep disturbance. For example, the notification source can be configured to impart vibrations unto a source of the snoring sound, responsive to the vibratory activation signal, to indicate the presence of the snoring condition.

### FIELD

Embodiments relate generally to electrical and electronic hardware, computer software, wired and wireless network communications, and wearable computing devices for sensing health and wellness-related physiological characteristics. More specifically, disclosed is an apparatus and method for snore detection and management implementing either wearable devices or non-wearable devices, or a combination thereof.

### BACKGROUND

Anomalies or disturbances in sleep (“sleep disturbances”) affect not only those persons experiencing a sleep disturbance during sleep, napping or resting, but also can affect other persons who also are also sleeping, resting, or otherwise wish not to be disturbed. Examples of sleep disturbances include snoring, sleep apnea, talking in one's sleep, night terrors (e.g., typically children who scream or otherwise cry), as well as health-related issues or disorders, such as complications that might lead to Sudden infant death syndrome (“SIDS”), and the like.

As an example, consider that snoring is not only an annoyance to people nearby, but snoring may be related to, or cause, a multitude of other health-related problems that range from feeling lousy after a night of poor sleep to hyperchlolesterolemia, sleep apnea, and tracheopharingeal infections. Snoring also may cause pain and discomfort that is detected after waking up (e.g., a sore throat). Of course, snoring can cause other people to lose sleep, thereby reducing their effectiveness.

Generally, snoring typically occurs in people during relatively non-REM deep sleep. Snoring arises due to muscles that relax during deep sleep (i.e., involuntary muscle relaxation), and cause the respiratory airways air to collapse. When a person breathes, the air is inhaled (or exhaled) and causes vibrations that gives rise to snoring sounds. Further, some people are more susceptible to snoring. For example, the likelihood that someone snores increases with certain factors, such as age, weight, and whether the person smokes. Generally, these factors relate to or affect the cross-sectional area of the airways, which may be constricted due to one or more of those factors.

Another example of a sleep disturbance, due to involuntary muscle relaxation, is bed wetting. Children that wet their beds learn to control their bladder sphincters thorough a largely unconscious process that comes about due to social pressure and shame. While wetting a bed has some built-in negative feedback mechanism that helps the subconscious mind of the affected person to learn not wet their bed, there are frequently very little effective techniques by which that a person receives feedback that they are snoring, without requiring another person to intervene. The intervening person then also loses sleep themselves. Unlike bed wetting, the long-term consequences of snoring can collectively take a toll in the health of the snorer.

Thus, what is needed is a solution for detecting sleep disturbances, such as snoring, by detecting and managing such sleep disturbances using either wearable devices or non-wearable devices, or a combination thereof, without the limitations of conventional techniques.

### BRIEF DESCRIPTION OF THE DRAWINGS

Various embodiments or examples (“examples”) of the invention are disclosed in the following detailed description and the accompanying drawings:

FIG. 1A illustrates an example of a variety of implementations of a wearable device, such as a wearable data-capable band, and a non-wearable device, according to some embodiments;

FIG. 1B depicts a block diagram of an example of an implementation of a media device of FIG. 1A, according to some embodiments;

FIG. 1C depicts a top view of a media device including a location determinator, according to some embodiments;

FIG. 1D depicts a perspective view of a media device including an example of an array of transducers, according to some embodiments;

FIG. 1E depicts a top view of a media device including another example of an array of transducers, according to some embodiments;

FIG. 2A illustrates an example of a specific implementation of a wearable device and a media device, according to some embodiments;

FIG. 2B illustrates another example of a specific implementation of a wearable device and a media device, according to some embodiments;

FIG. 3 depicts a wearable device including a skin surface microphone (“SSM”), in various configurations, according to some embodiments;

FIG. 4 is a diagram depicting examples of devices in which a microphone and/or a snore detector can be disposed in or distributed among, according to some examples;

FIG. 5A is a block diagram depicting a snore detector and a snore manager, according to some embodiments;

FIG. 5B depicts the generation of a window for validly detecting snoring sounds, according to some embodiments;

FIG. 6 depicts formation of an ad hoc network among wearable and non-wearable devices to address sleep disturbances, according to some embodiments;

FIG. 7 depicts implementation of at least a wearable device and a non-wearable device to detect and/or monitor sleep disturbances, as well as reducing the impact of such sleep disturbances, according to some embodiments;

FIG. 8 is an example flow diagram for detecting a snoring condition, according to some embodiments; and

FIG. 9 illustrates an exemplary computing platform disposed in a wearable device (or a non-wearable device) in accordance with various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

FIG. 1A illustrates an example of a variety of implementations of a wearable device, such as a wearable data-capable band, and a non-wearable device, according to some embodiments. Diagram 100 depicts a snore detector 122 and a snore manager 124, either of which (or both of which) can be disposed in one or more wearable devices and/or one or more non-wearable devices. In some example, components that constitute snore detector 122 and snore manager 124 can be distributed over any of the one or more wearable devices, the one or more non-wearable devices, and any other device not shown. Snore detector 122 is also configured to receive via path 109 acoustic energy or acoustic signals indicative of snoring sounds 103. Snore detector 122 is also configured to analyze sounds and detect the presence of a snoring condition (or any other sleep disturbance). Snore manager 124 is configured to determine that the condition of snoring (or another sleep disturbance) exists, and to cause generation of one or more signals to initiate actions, such as providing feedback, alerting other persons, memorializing or otherwise recording the various aspects of the snoring/other sleep disturbance to analyze at a later time, and other like actions. Note that FIG. 1A depicts an example in which a user or person is snoring, the disclosure is intended to be broad and non-limiting to detect and manage other sleep disturbances, such as those described herein.

According to some embodiments, snore detector 122 is configured to determine that a sound (e.g., acoustic energy propagating in a medium) is or likely is associated with a snoring sound 103. For example, snore detector 122 can be configured to receive an acoustic signal. An example of an “acoustic signal” can be sound or sound wave received, or an acoustic signal can be electrical signal representations of a sound (e.g., including data representing a sound), such as a snoring sound 103. In some examples, an acoustic signal is in an audible range of frequencies. In some embodiments, snore detector 122 can be configured to characterize the acoustic signal as a snoring sound 103 to determine presence of a snoring condition. In some examples, snore detector 122 can be configured to receive an acoustic signal via a transducer, to compare data representing characteristics of the acoustic signal with data representing criteria specifying sounds defining a snore, and to detect the presence of the snore condition upon a match between the data representing the characteristics of the acoustic signal and the data representing the criteria that can define the snore.

A snoring condition is a state of a user or person in which vibrations of respiratory structures during inhaling and exhaling air cause audible sounds to emit from the user or person. A snoring condition can be described as a sleep disturbance condition than includes any event in which either the user's sleep or others' sleep is impacted from such a condition. Examples of sleep disturbances can include snoring, sleep apnea, talking in one's sleep, night terrors (e.g., typically children who scream or otherwise cry), as well as health-related issues or disorders, such as complications that might lead to Sudden infant death syndrome (“SIDS”), and the like. Snore detector 122 is configured to differentiate snoring sounds from other types of sounds and to filter out non-related sources of noise. Further, snore detector 122 is configured to discriminate between snoring sounds produced by a wearer and other sounds (e.g., other snoring sounds) of someone else (e.g., a friend, spouse, partner, child, or the like). According to some embodiments, snore manager 124 is configured to determine that the condition of snoring (or another sleep disturbance) exists based on data received, for example, snore detector 122. Snore manager 124 is configured to cause generation of one or more signals to manage the snoring condition by, for example, causing initiation of one or more actions, including transmitting a notification signal to cause notification of the detection of the snoring sound. In various examples, the notification of the detection of the snoring sound can be directed to the person who is snoring, or to a person located within an audible range, or to any other person of interest.

In view of the foregoing, the functions and/or structures of snore detector 122 and snore manager 124, as well as their components, can facilitate the sensing of snoring conditions and can provide feedback to cease or reduce occurrences of such conditions or otherwise provide data that can improve the health of the person who is snoring. In some embodiments, real-time (or near real-time feedback) provided by snore detector 122 and snore manager 124 can provide relief to the snorer or to any affected persons nearby. For example, a person that is snoring can receive a notification (e.g., a haptic notification) that the person is associated with a snoring condition, and that person ought to take an action, such as change a sleeping position and/or effect conscious control of their breathing pattern to correct the situation. A combination of snore detector 122 and snore manager 124 can, at least in some cases, provide potential long-term effects of training the subconscious mind to stop snoring through repetition of notifications. Further, snore detector 122, as well as its components, can facilitate the identification of a source of a snoring sound 103. Snore detector 122 can identify a source of snoring, such as the identity of the person who is snoring. In some embodiments, snore detector 122 can be configured to identify a user (e.g., a person who snores) based on the acoustic characteristics of a sound that includes a snoring sound 103, whereby the characteristics of snoring sound 103 can be attributed to a specific user. According to some embodiments, snore detector 122 can be configured to identify a user based on data representing a location from which a snoring sound 103 emanates. By determining the occurrence of snoring, and the optional identification of the source of the snoring sound 103, snore manager 124 can be configured to determine one or more courses of action in which to take. In a first example, snore manager 124 can be configured to generate a notification signal to transmit to notification source, such as a vibratory energy source, to notify the person who is snoring that a snoring condition exists. That person can take any number of actions, such as rearranging a sleeping position to alleviate the condition. In a second example, snore manager 124 can be configured to generate a notification signal to another person (e.g., to a wearable device worn by another person) to alert that other person that a snoring condition (or any other sleep disturbance condition) exists for the person generating sounds related to a sleep disturbance. In a third example, snore manager 124 can be configured to cause generation of noise cancellation signals directed to one location to attenuate or otherwise reduce snoring sounds that are generated at another location, thereby providing, for example, a reduced impact to person(s) sleeping at one location when a person at another location is snoring.

A wearable device 104 can include snore detector 122 and snore manager 124, whereby detection of a sleep disturbance (e.g., a snoring sound) and snore management can be performed by or in a single wearable device, according to some embodiments. While wearable device 104 is shown worn about a wrist of a user 102, wearable device 104 is not so limited and can be worn, attached, or otherwise disposed adjacent to any limb or portion of user 102 suitable to at least detect snoring. An example of wearable device 104 can include one or more components of an UP™ band, or a variant thereof, manufactured by AliphCom, Inc., of San Francisco, Calif. In some embodiments, wearable device 104 can be configured to receive a notification signal, either from an internal or an external source, as a vibratory activation signal. Further, a vibratory energy source can be generated to impart vibrations unto a source of the snoring sound (e.g., a person who is snoring), responsive to the vibratory activation signal, to indicate the presence of the snoring condition. An example of a vibratory source of energy is described in U.S. patent application Ser. No. 13/180,320, filed on Jul. 11, 2011, which is incorporated by reference for all purposes.

As another example, a wearable device 105, such as wearable device 105a, can include snore detector 122 and/or snore manager 124. An example of wearable device 105a can include one or more components of a Jawbone ERA™ Blue Tooth® headset, or a variant thereof, manufactured by AliphCom, Inc., of San Francisco, Calif. In some embodiments, wearable device 104 and/or wearable device 105 can include structures and/or functionalities that constitute snore detector 122 and snore manager 124 or any portion thereof. Wearable device 105 can include a microphone 106 configured to contact (or to be positioned adjacent to) the skin of the wearer, whereby microphone 106 is adapted to receive sound and acoustic energy generated by the wearer (e.g., the source of snoring sound). Microphone 106 can also be disposed in wearable device 104. According to some embodiments, microphone 106 can be implemented as a skin surface microphone (“SSM”), or a portion thereof, according to some embodiments. An SSM can be an acoustic microphone configured to enable it to respond to acoustic energy originating from human tissue rather than airborne acoustic sources. As such, an SSM facilitates relatively accurate detection of physiological signals through a medium for which the SSM can be adapted (e.g., relative to the acoustic impedance of human tissue). Examples of SSM structures in which piezoelectric sensors can be implemented (e.g., rather than a diaphragm) are described in U.S. patent application Ser. No. 11/199,856, filed on Aug. 8, 2005, which is incorporated by reference. As used herein, the term human tissue can refer to, at least in some examples, as skin, muscle, blood, or other tissue. In some embodiments, a piezoelectric sensor can constitute an SSM. In at least one embodiment, snore detector 122 can transmit data 126 to media device 107 for further snore management processing. Data 126 can include acoustic signal information received from an SSM or other microphone, according to some examples. Data 126 can include acoustic-related information received from an SSM or other microphone, such as the amplitude of the snoring sound, according to some examples. In response, media device 107 can transmit data 130b including a notification signal and an amount of vibratory energy to impart. In some cases, the louder the snoring sound, the larger the amount of vibratory energy can be generated to notify person 102.

In yet another example, a non-wearable device 107 can be configured to implement at least a portion of any of snore detector 122 or at least a portion of snore manager 124. In at least one example, snore detector 122 and snore manager 124 are disposed within a non-wearable device 107. In some embodiments, wearable device 104 (or 105) and non-wearable device 107 can form a communication path 101 (e.g., to facilitate a wireless exchange of signals). In one example of an implementation, wearable device 104 can receive the acoustic signal and transmit data via path 146 representing the acoustic signal via path 101 to a non-wearable device 107, at which the acoustic signal is characterized to determine whether a sound is a snoring sound 103 associated with the presence of a snoring condition. Thereafter, non-wearable device 107 can transmit a notification signal 130b to cause notification of the detection of the snoring sound 103. Wearable device 104 then can receive notification signal 130b to generate vibrations to alert the wearer that he or she is snoring. An example of non-wearable device 107 can include wireless speakers and/or one or more components of a BIGJAMBOX™ or a JAMBOX™, or variants thereof, manufactured by AliphCom. Inc., of San Francisco, Calif.

In another example of an implementation, wearable device 104 can receive the acoustic signal and can be configured to characterize the acoustic signal to determine whether a sound is a snoring sound 103 associated with the presence of a snoring condition. Wearable device 104 can implement a snore manager 124 to initiate an action internally (e.g., generate vibrations) to notify the wearer via a notification signal 130a. Or, wearable device 104 can implement a snore manager 124 to cause non-wearable device 107 to initiate an action (e.g., alerting another wearer of wearable device 104 or generating noise cancellation signals). An example of a non-wearable device 107 is a media device, an example of which is described herein. In various embodiments, any partial or all functionalities of snore detector 122 and snore manager 124 can be implemented by or among any combination of wearable devices 104 or 105 and non-wearable device 107.

FIG. 1B depicts a block diagram of an example of some embodiments of a media device 107 of FIG. 1A having components including but not limited to a controller 151, a data storage (“DS”) system 103, an input/output (“I/O”) system 155, a radio frequency (“RF”) system 157, an audio/video (“A/V”) system 159, a power system 111, and a proximity sensing (“PROX”) system 113. A bus 110 is configured to facilitate communication among the controller 151, DS system 153, I/O system 155, RF system 157, AV system 159, power system 111, and proximity sensing system 113. Power bus 112 supplies electrical power from power system 111 to the controller 151, DS system 153, I/O system 155, RF system 157, AV system 159, and proximity sensing system 113.

Power system 111 may include a power source internal to the media device 158 such as a battery (e.g., AAA, AA batteries, or the like, including rechargeable batteries, such as a lithium ion or nickel metal hydride type battery, etc.) denoted as “BAT” 135. Power system 111 may be electrically coupled with a port 114 for connecting an external power source (not shown) such as a power supply that connects with an external AC or DC power source. Examples of power supplies include those that convert AC power to DC power, or convert AC power to AC power at a different voltage level. In other examples, port 114 may be a connector (e.g., an IEC connector) for a power cord that plugs into an AC outlet or other type of connecter, such as a universal serial bus (“USB”) connector. Power system 111 provides DC power for the various systems of media device 150. Power system 111 may convert AC or DC power into a form usable by the various systems of media device 150. Power system 111 may provide the same or different voltages to the various systems of media device 150. In applications where a rechargeable battery is used for BAT 135, the external power source may be used to power the power system 111, recharge BAT 135, or both. Further, power system 111 on its own or under control or controller 151 may be configured for power management to reduce power consumption of media device 150, by for example, reducing or disconnecting power from one or more of the systems in media device 150 when those systems are not in use or are placed in a standby or idle mode. Power system 111 may also be configured to monitor power usage of the various systems in media device 150 and to report that usage to other systems in media device 150 and/or to other devices (e.g., including other media devices 150) using one or more of the I/O system 155, RF system 157, and AV system 159, for example. Operation and control of the various functions of power system 111 may be externally controlled by other devices (e.g., including other media devices 150).

Controller 151 controls operation of media device 150 and may include a non-transitory computer readable medium, such as executable program code to enable control and operation of the various systems of media device 150. DS 153 may be used to store executable code used by controller 151 in one or more data storage mediums such as ROM, RAM, SRAM, RAM, SSD, Flash, etc., for example. Controller 151 may include but is not limited to one or more of a microprocessor (μP), a microcontroller (μP), a digital signal processor (DSP), an application specific integrated circuit (ASIC), as but a few examples. Processors used to implement controller 151 may include a single core or multiple cores (e.g., dual core, quad core, etc.). In some embodiments, controller 151 can be implemented in software as a virtual machine. Further, controller 151 can be implemented in hardware, software, or a combination thereof. Port 116 may be used to electrically couple controller 151 to an external device (not shown).

DS system 153 may include but is not limited to non-volatile memory (e.g., Flash memory), SRAM, DRAM, ROM, SSD, just to name a few. Media device 150, in at least in some implementation, can be designed to be compact, portable, or to have a small size footprint. In some cases, memory in DS 153 can be solid state memory (e.g., no moving or rotating components). Or, memory in DS 153 can include a hard disk drive (HDD) or a hybrid HDD. In some examples, DS 153 may be electrically coupled with a port 148 for connecting an external memory source (e.g., USB Flash drive, SD, SDHC, SDXC, microSD, Memory Stick, CF, SSD, etc.). Port 148 may be a USB or mini-USB port, or the like, for a Flash drive or a card slot for a Flash memory card or equivalent. In some examples, DS 153 includes data storage for configuration data, denoted as CFG 125, used by controller 151 to control operation of media device 150 and its various systems. DS 153 may include memory designate for use by other systems in media device 150 (e.g., MAC addresses for WiFi 141, network passwords, data for settings and parameters for A/V 159, and other data for operation and/or control of media device 150, etc.). DS 153 may also store data used as an operating system (OS) for controller 151. If controller 151 includes a DSP, then DS 153 may store data, algorithms, program code, an OS, etc. for use by the DSP, for example. In some examples, one or more systems in media device 150 may include their own data storage systems.

I/O system 155 may be used to control input and output operations between the various systems of media device 150 via bus 110 and between systems external to media device 150 via port 118. Port 118 may be a connector (e.g., USB, HDMI, Ethernet, fiber optic, Toslink, Firewire, IEEE 1394, or the like) or a hard-wired (e.g., captive) connection that facilitates coupling I/O system 155 with external systems. In some examples port 118 may include one or more switches, buttons, or the like, used to control functions of the media device 150 such as a power switch, a standby power mode switch, a button for wireless pairing, an audio muting button, an audio volume control, an audio mute button, a button for connecting/disconnecting from a WiFi network, an infrared (“IR”) transceiver, just to name a few. I/O system 155 may also control indicator lights, audible signals, or the like (not shown) that give status information about the media device 150, such as a light to indicate the media device 100 is powered up, a light to indicate the media device 100 is in wireless communication (e.g., WiFi, Bluetooth®, WiMAX, cellular, etc.), a light to indicate the media device 150 is Bluetooth® paired, in Bluetooth® pairing mode, Bluetooth® communication is enabled, a light to indicate the audio and/or microphone is muted, just to name a few. Audible signals may be generated by the I/O system 155 or via the AV system 159 to indicate status, etc. of the media device 150. Audible signals may be used to announce Bluetooth® status, powering up or down the media device 150, muting the audio or microphone, an incoming phone call, a new message such as a text, email, or SMS, just to name a few. In some examples, I/O system 155 may use optical technology to wirelessly communicate with other media devices 150 or other devices. Examples include but are not limited to infrared (“IR”) transmitters, receivers, transceivers, an IR LED, and an IR detector, just to name a few. I/O system 155 may include an optical transceiver OPT 185 that includes an optical transmitter 185t (e.g., an IR LED) and an optical receiver 185r (e.g., a photo diode). OPT 185 may include the circuitry necessary to drive the optical transmitter 185t with encoded signals and to receive and decode signals received by the optical receiver 185r. Bus 110 may be used to communicate signals to and from OPT 185. OPT 185 may be used to transmit and receive IR commands consistent with those used by infrared remote controls used to control AV equipment, televisions, computers, and other types of systems and consumer electronics devices. The IR commands may be used to control and configure the media device 150, or the media device 150 may use the IR commands to configure/re-configure and control other media devices or other user devices, for example.

RF system 157 includes at least one RF antenna 124 that is electrically coupled with a plurality of radios (e.g., RF transceivers) including but not limited to a Bluetooth® (BT) transceiver 120, a WiFi transceiver 141 (e.g., for wireless communications over a wireless and/or WiMAX network), and a proprietary Ad Hoc (AH) transceiver 140 pre-configured (e.g., at the factory) to wirelessly communicate with a proprietary Ad Hoc wireless network (e.g., AH-WiFi) (not shown). AH 140 and AH-WiFi are configured to allow wireless communications between similarly configured media devices (e.g., an ecosystem comprised of a plurality of similarly configured media devices) as will be explained in greater detail below. Note that an Ad Hoc wireless network need not be limited to WiFi and can implement any wireless networking protocol, regardless whether standardized or proprietary. RF system 157 may include more or fewer radios than depicted in FIG. 1B and the number and type of radios can be application dependent. Furthermore, radios in RF system 157 need not be transceivers, RF system 157 may include radios that transmit only or receive only, for example. Optionally, RF system 157 may include a radio 158 configured for RF communications using a proprietary format, frequency band, or other existent now or to be implemented in the future. Radio 158 may be used for cellular communications (e.g., 3G, 4G, or other), for example. Antenna 124 may be configured to be a de-tunable antenna such that it may be de-tuned 129 over a wide range of RF frequencies including but not limited to licensed bands, unlicensed bands, WiFi, WiMAX, cellular bands, Bluetooth®, from about 2.0 GHz to about 6.0 GHz range, and broadband, just to name a few. As will be discussed below, proximity sensing system 113 may use the de-tuning capabilities of antenna 124 to sense proximity of the user, other people, the relative locations of other media devices 150, just to name a few. Radio 158 (e.g., a transceiver) or other transceiver in RF system 157, may be used in conjunction with the de-tuning capabilities of antenna 124 to sense proximity, to detect and or spatially locate other RF sources such as those from other media devices 150, devices of a user, just to name a few. RF system 157 may include a port 123 configured to connect the RF system 157 with an external component or system, such as an external RF antenna, for example. The transceivers depicted in FIG. 1 are non-limiting examples of the type of transceivers that may be included in RF system 157. RF system 157 may include a first transceiver configured to wirelessly communicate using a first protocol, a second transceiver configured to wirelessly communicate using a second protocol, a third transceiver configured to wirelessly communicate using a third protocol, and so on. One of the transceivers in RF system 157 may be configured for short range RF communications, such as within a range from about 1 meter to about 15 meters, or less, for example. Another one of the transceivers in RF system 157 may be configured for long range RF communications, such any range up to about 50 meters or more, for example. Short range RF may include Bluetooth®, and near field communication (“NFC”) capabilities, for example; whereas, long range RF may include WiFi, WiMAX, cellular, for example.

AV system 159 includes at least one audio transducer, such as a loud speaker 160, a microphone 170, or both. AV system 159 further includes circuitry such as amplifiers, preamplifiers, or the like as necessary to drive or process signals to/from the audio transducers. Optionally, AV system 159 may include a display (“DISP”) 171, video device (“VID”) 172 (e.g., an image captured device or a web CAM, etc.), or both. DISP 171 may be a display and/or touch screen (e.g., a LCD, OLED, or flat panel display) for displaying video media, information relating to operation of media device 150, content available to or operated on by the media device 150, playlists for media, date and/or time of day, alpha-numeric text and characters, caller ID, file/directory information, a GUI, just to name a few. A port 122 may be used to electrically couple AV system 159 with an external device and/or external signals. Port 122 may be a USB, HDMI, Firewire/IEEE-1394, 3.5 mm audio jack, or other. For example, port 122 may be a 3.5 mm audio jack for connecting an external speaker, headphones, earphones, etc. for listening to audio content being processed by media device 150. As another example, port 122 may be a 3.5 mm audio jack for connecting an external microphone or the audio output from an external device. In some examples, SPK 160 may include but is not limited to one or more active or passive audio transducers such as woofers, concentric drivers, tweeters, super tweeters, midrange drivers, sub-woofers, passive radiators, just to name a few. As such, SPK 160 make include an array of transducers configurable to localize sound at a focal point to deliver sound (or “anti-sound”) to a person at a location including the focal point. “Anti-sound” can refer to the creation of one or more sound beams representing noise cancellation signals that are configured to generate one or more nulls to reduce, for example, snoring sounds at the focal point.

MIC 170 may include one or more microphones and the one or more microphones may have any polar pattern suitable for the intended application including but not limited to omni-directional, directional, bi-directional, uni-directional, bi-polar, uni-polar, any variety of cardioid pattern, and shotgun, for example. MIC 170 may be configured for mono, stereo, or other. MIC 170 may be configured to be responsive (e.g., generate an electrical signal in response to sound) to any frequency range including but not limited to ultrasonic, infrasonic, from about 20 Hz to about 20 kHz, and any range within or outside of human hearing. In some applications, the audio transducer of AV system 159 may serve dual roles as both a speaker and a microphone. In some examples, MIC 170 can represent an array of microphones configured to detect sounds from different locations (e.g., different sectors or angular areas) about media device 150. For example, different microphones in an array can be configured to pick up acoustic signals in specific directions or ranges of direction (e.g., over a specific angle or arc). Such microphones can be unidirectional or “shot gun” like in structure or functionality, and can be implemented in hardware, software, or a combination thereof.

Circuitry in AV system 159 may include but is not limited to a digital-to-analog converter (“DAC”) and algorithms for decoding and playback of media files such as MP3, FLAC, AIFF, ALAC, WAV, MPEG, QuickTime, AVI, compressed media files, uncompressed media files, and lossless media files, just to name a few, for example. A DAC may be used by AV system 159 to decode wireless data from a user device or from any of the radios in RF system 157. AV system 159 may also include an analog-to-digital converter (“ADC”) for converting analog signals, from MIC 170 for example, into digital signals for processing by one or more system in media device 150.

Media device 150 may be used for a variety of applications including but not limited to wirelessly communicating with other wireless devices, other media devices 150, wireless networks, and the like for playback of media (e.g., streaming content), such as audio, for example. The actual source for the media or audio need not be located on a user's device (e.g., smart phone, MP3 player, iPod™, iPhone™, iPad™, Android™, laptop, PC, etc.). For example, media files to be played back on media device 150 may be located on the Internet, a web site, or in the cloud, and media device 150 may access (e.g., over a WiFi network via WiFi 141) the files, process data in the files, and initiate playback of the media files. Media device 150 may access or store in its memory a playlist or favorites list and playback content listed in those lists. In some applications, media device 150 will store content (e.g., files) to be played back on the media device 150 or on another media device 150. In some embodiments, media device 150 is configured to operate on snoring sounds as audio, with which actions can be taken responsive to detection of such snoring sounds or sleep disturbances.

Media device 150 may include a housing, a chassis, an enclosure or the like, denoted in FIG. 1B as 199. The actual shape, configuration, dimensions, materials, features, design, ornamentation, aesthetics, and the like of housing 199 will be application dependent and a matter of design choice. Therefore, housing 199 need not have the rectangular form depicted in FIG. 1B or the shape, configuration etc., depicted in the Drawings of the present application. Housing 199 can be composed of one or more structural elements, and housing 199 may be comprised of several housings that form media device 150. While in some embodiments, housing 199 is configured to be non-wearable, other embodiments can provide that housing 199, as well as media device 107, can be configured to be worn, mounted, or otherwise connected to or carried by a human being. Therefore, at least one example of media device 107 of FIG. 1A can implemented as a wearable device. For example, housing 199 may be configured as a wristband, an earpiece, a headband, a headphone, a headset, an earphone, a hand held device, a portable device, a desktop device, an accessory to attach to any other portions of wearable items, or the like.

In other examples, housing 199 may be configured as speaker, a subwoofer, a conference call speaker, an intercom, a media playback device, just to name a few. If configured as a speaker (e.g., an audio source, for audio notifications or for noise cancellation), then the housing 199 may be configured as a variety of speaker types including but not limited to an array of transducers, a left channel speaker, a right channel speaker, a center channel speaker, a left rear channel speaker, a right rear channel speaker, a subwoofer, a left channel surround speaker, a right channel surround speaker, a left channel height speaker, a right channel height speaker, any speaker in a 3.1, 5.1, 7.1, 9.1 or other surround sound format, without being limited to surround sound formats, including those having two or more subwoofers or having two or more center channels, for example. In other examples, housing 199 may be configured to include a display (e.g., DISP 171) for viewing video, serving as a touch screen interface for a user, providing an interface for a GUI, for example.

Proximity sensing system 113 may include one or more sensors denoted as SEN 195 that are configured to sense 197 an environment 198 external to the housing 199 of media device 150. Using SEN 195 and/or other systems in media device 150 (e.g., antenna 124, SPK 160, MIC 170, etc.), proximity sensing system 113 senses 197 an environment 198 that is external to the media device 150 (e.g., external to housing 199). proximity sensing system 113 may be used to sense one or more of proximity of the user or other persons to the media device 150 or other media devices 150. Proximity sensing system 113 may use a variety of sensor technologies for SEN 195 including but not limited to ultrasound, infrared (IR), passive infrared (PIR), optical, acoustic, vibration, light, RF, temperature, capacitive, inductive, just to name a few. Proximity sensing system 113 may be configured to sense location of users or other persons, user devices, and other media devices 150, without limitation. Output signals from proximity sensing system 113 may be used to configure media device 150 or other media devices 150, to re-configure and/or re-purpose media device 150 or other media devices 150 (e.g., change a role the media device 150 plays for the user, based on a user profile or configuration data), just to name a few. A plurality of media devices 150 in an eco-system of media devices 150 may collectively use their respective proximity sensing system 113 and/or other systems (e.g., RF 157, de-tunable antenna 124, AV 159, etc.) to accomplish tasks including but not limited to changing configuration, re-configuring one or more media devices, implement user specified configurations and/or profiles, insertion and/or removal of one or more media devices in an eco-system, just to name a few.

According to some embodiments, snore detector 122 and/or snore manager 124 of FIG. 1A, and one or more of their components, can be implemented in media device 150 FIG. 1B. Controller 151 can be configured to execute instructions in data storage 153 to provide for the functionality of snore detector 122 and/or snore manager 124. But note that snore detector 122 and/or snore manager 124 are not limited to only implementations as algorithms.

FIG. 1C depicts a top view of a media device 107 of FIG. 1A or 1B including a location determinator, according to some embodiments. In this example, diagram 180 depicts a media device 181a including a location determinator 187 and an array of microphones 183 each being configured to detect or pick-up sounds originating at a location. Location determinator 187 can be configured to receive acoustic signals from each of the microphones or directions from which a sound, such as a snoring sound, originates. For example, a first microphone can be configured to receive sound 184a originating from a sound source at location (“1”) 182a, whereas a second microphone can be configured to receive sound 184b originating from a sound source at location (“2”) 182b. For example, location determinator 187 can be configured to determine the relative intensities or amplitudes of the sounds received by a subset of microphones and identify the location (e.g., direction) of a sound source based on a corresponding microphone receiving, for example, the greatest amplitude. In some cases, a location can be determined in three-dimensional space. Location determinator 187 can be configured to calculate the delays of a sound received among a subset of microphones relative to each other to determine a point (or an approximate point) from which the sound originates. Delays can represent farther distances a sound travels before being received by a microphone. By comparing delays and determining the magnitudes of such delays, in, for example, an array of transducers operable as microphones, the approximate point from which the sound originates can be determined. In some embodiments, location determinator 187 can be configured to determine the source of sound by using known time-of-flight and/or triangulation techniques and/or algorithms.

FIG. 1D depicts a perspective view of a media device including an example of an array of transducers, according to some embodiments. In this example, a media device 181b includes an example array of transducers 186, which can include any type of transducer in which at least one type of transducer is configured to receive or transmit sounds in a range of frequencies. The array of transducers 186 can be linearly arranged or can be disposed in any other arrangement, and need not be limited to one linear arrangement.

FIG. 1E depicts a top view of a media device including another example of an array of transducers, according to some embodiments. In this example, diagram 190 depicts a media device 191a includes an example array of transducers 192, which can include any type of transducer in which at least one type of transducer is configured to receive or transmit sounds in a range of frequencies. Media device 191a is shown to include a location determinator (“LD”) 187 configured to determine an approximate location or direction 182c from a source sound originates, and a multiple mode (“MM”) manager 189 configured to manage modes of operation of the array of transducers in multiple modes. For example, one or more transducers 192 can operate as a microphone in a first mode, and one or more transducers 192 can operate as a speaker in a second mode. In at least some embodiments, one or more transducers 192 can operate as a speaker to propagate noise cancellation signals to form one or more nulls 195 at a second location 183d to reduce or negate the impact of the sounds (e.g., snoring sounds generated at location 182c) at second location 183d, which can include another person who might otherwise hear the snoring sound. Note that some transducers 192 can operate as microphones in one mode and other transducers 192 can operate as speakers in another mode, whereby the two modes can overlap for at least a period of time.

To illustrate, consider that a first person is located at location 182c and a second person is located at location 183d. In some embodiments, media device 191 and location determinator 187 are configured to determine location 182c based on snoring sounds received into the array of transducers 192 from the first person, and determines the location 183d based on sleeping sounds (e.g., non-snoring sounds, including exhaling and inhaling deeply, sounds emitted by changes positions in bed, mattress spring squeaks, etc.) received into the array of transducers 192 from the second person. In this example, multiple mode manager 189 is configured to operate one or more transducers 192 in the array as microphones to receive the above-described sounds. For example, transducer 194a can receive a snoring sound via path 193a and transducer 194b can receive the snoring sound via path 193b. As there are different amplitudes and/or delays associated with the paths, location determinator 187 can determine location 182c. In some embodiments, one or more transducers 192 in the array are configured by multiple mode manager 189 in a second mode to generate audio, and more specifically, noise cancellation signals to create one or more nulls 195 at location 183d to reduce the snoring sound amplitudes received by the second person. Note that if the second person becomes a source of snoring sounds, then multiple mode manager 189 can configure one or more transducers 192 in the array to generate one or more nulls at location 182c (not shown).

FIG. 2A illustrates an example of a specific implementation of a wearable device and a media device, according to some embodiments. Diagram 200 depicts a snore detector 122 and a snore manager 124, both of which are disposed in this example in media device 207. In the example shown, a person 202 who is snoring can generate snoring sounds 203 (e.g., as acoustic signals). Snoring sounds 203 is received via path 209 (e.g., into a microphone) and a snoring condition is detected by snore detector 122. Snore detector 122 transmits an indication of the snoring condition to snore manager 124, which, in turn, generates a notification signal 230b. Notification signal 230b is transmitted (e.g., wirelessly) to wearable device 204, and in response, wearable device 204 generates vibrations to notify person 202 that a snoring condition is present. In some cases, person 202 can take an action, such as re-positioning themselves to stop the snoring sounds.

FIG. 2B illustrates another example of a specific implementation of a wearable device and a media device, according to some embodiments. As shown, a first person 202a is wearing a wearable device 204a in a location 282a, and a second person 202b is disposed in a location 282b including a media device 207a. In this example, media device 207a is configured to detect sounds associated with a sleep disturbance associated with person 202b, and to transmit a notification signal 230c to wearable device 204a, which, in response, generates vibratory energy as a haptic signal for imparting upon person 202a (or any other signal to cause visual or audible notifications). Once alerted, person 202a can address the sleep disturbance associated with person 202b. In some examples, person 202b is a baby and person 202a is an adult, whereby media device 207a is configured to detect sound (or lack of sound). Location 282a and location 282b can be different rooms in which sleep disturbance sounds are attenuated such that person 202a, when asleep, cannot readily hear or become aware of the sleep disturbance condition. A sound associated or otherwise characterized as a sleep disturbance can be detected from the baby by media device 207a, which, in turn, notifies the parent of the sleep disturbance. Other applications are possible. For example, person 202b can be a patient and person 202a can be a care-giver. For example, a snore detector implemented in media device 207a (or in a wearable device 204a or the like) can be configured to detect sleep disturbances, such as sleep apnea, and associated sounds. Sounds 290 are examples of period of time 291 in which apnea occurs between two breathing cycles 292a and 292b, which typically have larger amplitudes than normal snoring sounds. As such, detection of sleep apnea can be a function of an amount time 191 (e.g., 13 seconds or more) during which no normative snoring is detected, and also a function of the detection of snoring having larger amplitudes than normal snoring amplitudes. In one embodiment, a snore manager is configured to record the apneic events for analysis and reporting to the user to ensure health is maintained and any indications of apnea are documented.

FIG. 3 depicts a wearable device including a skin surface microphone (“SSM”), in various configurations, according to some embodiments. Diagram 300 of FIG. 3 depicts a wearable device 301, which has an outer surface 302 and an inner surface 304. In some embodiments, wearable device 301 includes a housing 303 configured to position a sensor 310a (e.g., an SSM including, for instance, a piezoelectric sensor or any other suitable sensor) to receive an acoustic signal originating from human tissue, such as skin surface 305. As shown, at least a portion of sensor 310a can be formed external to surface 304 of wearable housing 303. The exposed portion of the sensor can be configured to contact skin 305. In some embodiments, the sensor (e.g., SSM) can be disposed at position 310b at a distance (“d”) 322 from inner surface 304. Material, such as an encapsulant, can be used to form wearable housing 303 to reduce or eliminate exposure to elements in the environment external to wearable device 301. In some embodiments, a portion of an encapsulant or any other material can be disposed or otherwise formed at region 310a to facilitate propagation of an acoustic signal to the piezoelectric sensor. The material and/or encapsulant can have an acoustic impedance value that matches or substantially matches the acoustic impedance of human tissue and/or skin. Values of acoustic impedance of the material and/or encapsulant can be described as being substantially similar to the human tissue and/or skin when the acoustic impedance of the material and/or encapsulant varies no more than 60% of that of human tissue or skin, according to some examples.

Examples of materials having acoustic impedances matching or substantially matching the impedance of human tissue can have acoustic impedance values in a range that includes 1.5×10⁶ Pa×s/m (e.g., an approximate acoustic impedance of skin). In some examples, materials having acoustic impedances matching or substantially matching the impedance of human tissue can provide for a range between 1.0×10⁶ Pa×s/m and 1.0×10⁷ Pa×s/m. Note that other values of acoustic impedance can be implemented to form one or portions of housing 303. In some examples, the material and/or encapsulant can be formed to include at least one of silicone gel, dielectric gel, thermoplastic elastomers (TPE), and rubber compounds, but is not so limited. As an example, the housing can be formed using Kraiburg TPE products. As another example, housing can be formed using Sylgard® Silicone products. Other materials can also be used.

Further to FIG. 3, wearable device 301 also includes a snore detector 322, a snore manager 324, a vibratory energy source 328, and a transceiver 326. Snore detector 322 can be configured to receive acoustic signals either from sensor 310a or a sensor at location 310b via acoustic impedance-matched material. Upon detecting a snoring condition, snore detector 322 communicates the condition to snore manager 324, which, in turn, generates a notification signal as a vibratory activation signal, thereby causing vibratory energy source (e.g., mechanical motor as a vibrator) to impart vibration through housing 303 unto a source of the snoring sound, responsive to the vibratory activation signal, to indicate the presence of the snoring condition. Also, wearable device 301 can optionally include a transceiver 326 configured to transmit signal 319 as a notification signal via, for example, an RF communication signal path. In some examples, transceiver 326 can be configured to transmit signal 319 to include data representative of the acoustic signal received from sensor 310, such as an SSM. Thus, the snoring sound as received from an SSM in wearable device 301 can be transmitted to a media device for further processing (e.g., noise cancellation based on signal 319 including data representing acoustic signals picked up at the SSM).

FIG. 4 is a diagram depicting examples of devices in which a microphone, such as an acoustic sensor, and/or a snore detector can be disposed or distributed among, according to some examples. Diagram 400 depicts examples of devices (e.g., wearable or carried) in which snore detector 420 and/or acoustic sensor 210 (e.g., an SSM) can be disposed, but those devices are not limited to, a mobile phone 480, a headset 482, eyewear 484, and a wrist-based wearable device 470 (e.g., a wrist watch-like wearable computing device). In some instances, snore detector 420 and/or acoustic sensor 410 can be implemented as, or in operation with, an acoustic sensor 421 or 422. For example, acoustic sensor 421 can be disposed on or at an earloop 423 of headset 482 (e.g., a Wi-Fi or Bluetooth® communications headset) to position acoustic sensor 410 adjacent to human tissue (e.g., behind or internal to an ear). Or, acoustic sensor 421 can be disposed in or at the ear bud configured to be inserted into the ear canal. Acoustic sensor 422 is disposed on or at the ends of eyewear 484 (e.g., at temple tips that extend over an ear) to position acoustic sensor 410 adjacent to human tissue (e.g., behind or internal to an ear). Acoustic sensors, such as sensor 422, can be configured to detach and attach, as shown in view 454, to any of the devices described. Further, acoustic sensors described in FIG. 4 can include a transceiver to establish communications links 452 (e.g., wireless or acoustic data links) to communicate sleep disturbance-related data signals among the devices.

FIG. 5A is a block diagram depicting a snore detector and a snore manager, according to some embodiments. As shown in diagram 500, snore detector 522 includes an acoustic matcher 523, a repository 526, an acoustic characterizer 530, which is optional, a user characterizer 544, a snore indicator 540, a window determinator 542, a timer 545, which can be optional, and a motion analyzer 546, which can be optional. Snore detector 522 is configured to receive acoustic signals 508, such as acoustic signals received from an SSM. Acoustic signals 508 can include snoring sounds 501, which can be represented by an amplitude (“A”) 516 and by time-related characteristics (e.g., a time interval 514 between snoring sounds) for a specific snoring sound 512. As respirational structures and user characteristics vary from person-to-person, snoring sounds 512 can be unique to an individual, and, thus, can be used to identify a person who is snoring (i.e., snoring sound 512 can be used as an audible “finger print” that identifies a snorer). To either identify the person snoring or detect a snoring sound relative to other types of sounds, or both, acoustic matcher 523 receives the acoustic signal, such as snoring sounds 501, and compares the received acoustic signal against data representing characteristics of the acoustic signal to data representing criteria specifying sounds defining a snore. In this example, data representing criteria specifying sounds defining a snore is stored in repository 526. An example of the criteria can be data 527 representing snoring sound profiles describing, for example, the amplitudes, timing, durations, and general sound wave shapes for a particular person who is snoring. Such data can be captured using an acoustic characterizer 530, which can be used to characterize the sounds of a particular person as a snoring sound. For example, acoustic characterizer 530 can capture data 527 when only sounds of the particular person during sleep are available to form data 527. Acoustic characterizer 530 can capture data 527 from sounds received only from different people (e.g., at different times). Then, data 527 can be used to detect the identity of the snorer as well as differentiate that person's snoring sounds from other sounds, including other persons' snoring sounds. Criteria can include any type of data 528, such as spectral energy, frequency ranges, etc., that can be used to describe a snoring sound for purposes of at least differentiating a snore from other sounds.

Once acoustic matcher 523 matches received acoustic signals with criteria defining a snoring, at least within a range of tolerance (e.g., up to 40% deviation from what is expected, for at least one criterion, such as amplitude). The range of tolerance represents allowable deviation of snoring sounds from criteria for data 527 representing snoring sound profiles, while still indicating a snoring condition is present. In some embodiments, snore indicator 540 generates an indication of a snoring condition during a “window” (i.e., a window of validity) of a sleep cycle in which snoring sounds are likely, thereby filtering out sounds that are not likely snoring sounds. Window determinator 542 is configured to determine windows in which to validate an indication of a snoring condition. A window can be established based on a user characterizer 544, a timer 545, and/or a motion analyzer 546. User characterizer 544 is configured to characterize the acoustic signal as the snoring sound based on receiving data representing characteristics of a user associated with the snoring condition. For example, user characteristics can include one or more of an age, a height, a weight, a body fat percentage, and an indication whether the user smokes. As these factors relate to or affect the cross-sectional area of the airways, the presence of one or more of those factors (and the degree or magnitude of such factors) can predict the likelihood that an acoustic signal is a snoring sound. Upon determining that the data representing the characteristics of the user is indicative of the presence of the snoring condition, user characterizer 544 can enable characterization of the acoustic signal as the snoring sound (e.g., by providing a window as generated by window determinator 542). Therefore, to illustrate, consider that a first acoustic signal may be deemed a snoring sound, if produced by an overweight person that smokes and drinks alcohol. By contrast, another similar acoustic signal may not be deemed a snoring sound for a person having a normal height-to-weight proportion and does not smoke or drink.

In another embodiment, a motion analyzer 546 is configured to determine whether an acoustic signal is likely a snoring sound based on motion of the person who is subject to snoring conditions. Normal snoring typically occurs more frequently during deep sleep (e.g., stage 4) and is not likely to occur during REM sleep. Further, motion is generally non-existent during REM sleep as muscles can be immobilized. Thus, motion in REM sleep is generally less than at other stages of sleep. Given this, motion analyzer 546 can analyze motion data from a motion sensor 555, such as an accelerometer. As such, motion analyzer 546, upon detecting motion, can be configured to receive data representing an amount of motion that is substantially coextensive with the snoring sound. Based on the amount of motion, motion analyzer 546 can be configured to determine that the analyzed motion is associated with motion that can exist during a snoring condition, and then can enable characterization of the acoustic signal as the snoring sound. For example, motion analyzer 546 can be configured to determine that relatively no or little motion can be associated with lack of motion during REM, thereby indicating that snoring is less likely to occur, thereby preventing an indication of a snoring condition from being validated. In some embodiments, different ranges of motion can be associated (e.g., empirically or by prediction) with different stages of sleep. As such, motion analyzer 546 can determine one or more stages of sleep, and then can determine the validity of a sound as a snoring sound based on the level or amount of motion detected by motion sensor 555, which can be disposed in a wearable device. In other embodiments, a timer 545 is configured to facilitate a window during which snoring sound data is validated based on approximate reoccurring times in one or more sleep cycles when snoring is likely to occur. Given the above-described functionality, window determinator 542 is configured to validate snoring indication data provided by snore indicator 540 via path 541 to snore manager 524. As such, window determinator 542 can validate sounds and acoustic signals as snoring sounds based on data generated by one or more of a user characterizer 544, a timer 545, and/or a motion analyzer 546.

Snore manager 524 includes a source identifier 547, a location determinator 548, and a mode manager 549. Source identifier 547 is configured to receive data representing the identity of the person who is snoring via path 543, based on determining a match between received acoustic signals and criteria defining snoring sounds, which can uniquely associated with a specific person. Snore manager 524 can transmit the identity via transmitter 550, which can be an RF transceiver, as snore-related data 552. Other devices, such as media devices, can use this information to alert other persons to the identity of a person that is snoring. Snore manager 524 is configured to send an activation signal to notification source 560, which can be configured to generate vibratory energy. Notification source 560 is not limited to generating vibratory energy, but, in other examples, can be configured to generate audio (e.g., via a speaker as an alert) and lighting effects (e.g., via one or more LEDs or other lights disposed in a media device). Location determinator 548, in some embodiments, can determine the location of the snoring sound origination, and if the person's identity associated with the location is known, then location determinator 548 can determine the identity of the snorer. Otherwise, location determinator 548 can determine a location of a snoring sound as described herein. Mode manager 549 is configured to generate noise cancellation signals in at least one mode by controlling noise cancellation signal generator 579, which is configured to control an array of transducers (not shown). In some embodiments, noise cancellation signal generator 579 is configured to generate sound waves or sound beams with equivalent magnitudes of the snoring sounds, but with the phases of the generated sound waves being inverted to combine to form a new wave, or a null, whereby the snoring sound is effectively canceled or reduced at a particular location.

FIG. 5B depicts the generation of a window of validity for detecting snoring sounds, according to some embodiments. Consider in diagram 560 that a person who is sleeping passes through one or more sleep cycles over a duration 1551 between a sleep start time 1550 and sleep end time 1552. There is a general reduction of motion when a person passes from a wakefulness state 1542 into the stages of sleep, such as into light sleep 1546 in duration 1554. Motion indicative of “hypnic jerks” or involuntary muscle twitching motions typically occur during light sleep state 1546. The person then passes into a deep sleep state 1548 and a REM state 1544 for durations 1555 and 1553, respectively. In a deep sleep state 1548, a person has a decreased heart rate and body temperature, with the absence voluntary muscle motions to confirm or establish that a user is in a deep sleep state. The person then passes into REM sleep during which muscles are immobile. As shown, window determinator is configured to generate a window 561 during at least deep sleep durations 1555 in which to validate that snoring sounds 580, such as snoring sounds 582. Otherwise, sounds outside window 561, such as sound 584, are not validated, and thus, are not analyzed as snoring sounds.

FIG. 6 depicts formation of an ad hoc network among wearable and non-wearable devices to address sleep disturbances, according to some embodiments. Diagram 600 depicts a user 602a disposed at location 601a and a user 602b disposed at location 601b. Users 602a and 602b can generate snoring sounds at sources 606a and 606b of snoring sounds, respectively. Further, users 602a and 602b can wear wearable devices 604a and 604b, respectively. As shown, wearable devices 604a and 604b can form an ad hoc network 603a including wireless communication paths 655 that include a media device 620, which includes at least a microphone 622 and array of transducers 624 (e.g., as speakers). Notification signals 610 and other data can be exchanged via ad hoc network 603a.

FIG. 7 depicts implementation of at least a wearable device and a non-wearable device to address sleep disturbances, according to some embodiments. Diagram 700 depicts a user 702a disposed at location 701a and a user 702b disposed at location 701b. Users 702a and 702b can generate snoring sounds at sources 706a and 706b of snoring sounds, respectively. Users 702a and 702b can generate other sounds, like normal sleep sounds or other sound related to other sleep disturbances, too. Further, users 702a and 702b can wear wearable devices 704a and 704b, respectively. As shown, wearable devices 704a and 704b can form an ad hoc network of wireless communication paths that include a media device 720, which, in turn, includes at least a microphone 722 and an array of transducers 724 (e.g., as two or more speakers). In the example shown, user 702a and its source 706a of sounds are generating snoring sounds 703a directed to media device 720 and snoring sounds 703b directed to user 702b. In one instance, media device 702 is configured to receive via microphone 722 snoring sounds 703, and, in response, generate noise cancellation signals 712 configured to cancel or reduce snoring sounds 703b that impinge upon user 702b at location 701b. In another instance, media device 702 is configured to receive via a wireless signal data 710 representing snoring sounds 703 that, for example, are sensed via an SSM in wearable device 704a. In response, media device 702 is configured to generate noise cancellation signals 712 that are configured to cancel or reduce snoring sounds 703b that otherwise might impinge upon user 702b at location 701b. In various embodiments, one or more media devices 720 can be disposed at one or more positions 730a, 730b, and 730c to enhance noise cancellation.

FIG. 8 is an example flow diagram for detecting a snoring condition, according to some embodiments. At 802, flow 800 begins with receiving an acoustic signal. At 804, an acoustic signal is characterized to determine the presence of snoring. At 806, a determination is made as to whether the source of snoring is to be identifies. If so, the source of the snoring is identified at 807, and flow 800 moves to 808. Otherwise, flow 800 moves to 808 to identify locations that can include the source of snoring sounds. At 808, a determination is made as to whether to identify locations. If so, the locations of the snoring are identified at 809, and flow 800 moves to 810. Otherwise, flow 800 moves to 810 to initiate notification via generation of a notification signal. At 812, vibratory energy is generated to emit vibrations. At 816, a determination is made as to whether flow 800 is terminated.

FIG. 9 illustrates an exemplary computing platform disposed in a wearable device (or a non-wearable device) in accordance with various embodiments. In some examples, computing platform 900 may be used to implement computer programs, applications, methods, processes, algorithms, or other software to perform the above-described techniques. Computing platform 900 includes a bus 902 or other communication mechanism for communicating information, which interconnects subsystems and devices, such as one or more processors 904, system memory 906 (e.g., RAM, etc.), storage device 908 (e.g., ROM, etc.), a communication interface 913 (e.g., an Ethernet or wireless controller, a Bluetooth controller, etc.) to facilitate communications via a port on communication link 921 to communicate, for example, with a computing device, including mobile computing and/or communication devices with processors. Processor 904 can be implemented with one or more central processing units (“CPUs”), such as those manufactured by Intel® Corporation, or one or more virtual processors, as well as any combination of CPUs and virtual processors. Computing platform 900 exchanges data representing inputs and outputs via input-and-output devices 901, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 900 performs specific operations by processor 904 executing one or more sequences of one or more instructions stored in system memory 906, and computing platform 900 can be implemented in a client-server arrangement, peer-to-peer arrangement, or as any mobile computing device, including smart phones and the like. Such instructions or data may be read into system memory 906 from another computer readable medium, such as storage device 908. In some examples, hard-wired circuitry may be used in place of or in combination with software instructions for implementation. Instructions may be embedded in software or firmware. The term “computer readable medium” refers to any tangible medium that participates in providing instructions to processor 904 for execution. Such a medium may take many forms, including but not limited to, non-volatile media and volatile media. Non-volatile media includes, for example, optical or magnetic disks and the like. Volatile media includes dynamic memory, such as system memory 906.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 902 for transmitting a computer data signal.

In some examples, execution of the sequences of instructions may be performed by computing platform 900. According to some examples, computing platform 900 can be coupled by communication link 921 (e.g., a wired network, such as LAN, PSTN, or any wireless network) to any other processor to perform the sequence of instructions in coordination with (or asynchronous to) one another. Computing platform 900 may transmit and receive messages, data, and instructions, including program code (e.g., application code) through communication link 921 and communication interface 913. Received program code may be executed by processor 904 as it is received, and/or stored in memory 906 or other non-volatile storage for later execution.

In the example shown, system memory 906 can include various modules that include executable instructions to implement functionalities described herein. In the example shown, system memory 906 includes a snore detector module 954 configured to implement a motion analyzer module 965 and a user characterizer module 956, and also includes a snore manager module 955 configured to implement a source identifier module 957 and a mode manager module 959, any of which can be configured to provide one or more functions described herein.

Wearable devices and non-wearable devices can be in communication (e.g., wired or wirelessly) with a mobile device, such as a mobile phone or computing device. In some cases, a mobile device, or any networked computing device (not shown) in communication with a wearable device or mobile device, can provide at least some of the structures and/or functions of any of the features described herein. As depicted in the figures above, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1A (or any subsequent figure) can represent one or more algorithms. Or, at least one of the elements can represent a portion of logic including a portion of hardware configured to provide constituent structures and/or functionalities.

For example, snore detector 522 of FIG. 5A and any of its one or more components can be implemented in one or more computing devices (i.e., any mobile computing device, such as a wearable device or mobile phone, whether worn or carried) that include one or more processors configured to execute one or more algorithms in memory. Also, snore detector 524 of FIG. 5A and any of its one or more components can be implemented in one or more computing devices (i.e., any mobile computing device, such as a wearable device or mobile phone, whether worn or carried) that include one or more processors configured to execute one or more algorithms in memory. Thus, at least some of the elements in described in any figure can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. Thus, at least one of the elements in any figure can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. A method comprising:
receiving an acoustic signal;
characterizing the acoustic signal as a snoring sound to determine presence of a snoring condition;
transmitting a notification signal to cause notification of the detection of the snoring sound;
receiving the notification signal as a vibratory activation signal; and
causing a vibratory energy source to impart vibrations unto a source of the snoring sound, responsive to the vibratory activation signal, to indicate the presence of the snoring condition.

2. The method of claim 1, wherein characterizing the acoustic signal as the snoring sound comprises:
receiving data representing an amount of motion substantially coextensive with the snoring sound;
determining the amount of motion is association with the snoring condition; and
enabling characterization of the acoustic signal as the snoring sound.

3. The method of claim 1, wherein characterizing the acoustic signal as the snoring sound comprises:
receiving data representing characteristics of a user associated with the snoring condition;
determining that the data representing the characteristics of the user is indicative of the presence of the snoring condition; and
enabling characterization of the acoustic signal as the snoring sound.

4. The method of claim 3, wherein receiving the data representing the characteristics of the user comprises:
receiving data representing one or more of an age, a height, a weight, a body fat percentage, and an indication whether the user smokes.

5. The method of claim 1, wherein characterizing the acoustic signal as the snoring sound comprises:
receiving the acoustic signal via a transducer;
comparing data representing characteristics of the acoustic signal to data representing criteria specifying sounds defining a snore; and.
detecting the presence of the snore condition upon a match between the data representing the characteristics of the acoustic signal and the data representing the criteria that define the snore.

6. The method of claim 5, wherein receiving the acoustic signal via the transducer comprises:
receiving the acoustic signal via a skin surface microphone (“SSM”) in a wearable device.

7. The method of claim 6, wherein receiving the acoustic signal via the SSM comprises:
receiving the acoustic signal via a portion of a housing for the wearable device including material having an impedance substantially similar to the impedance of skin.

8. The method of claim 1, wherein receiving the acoustic signal comprises:
receiving the acoustic signal via an SSM in a wearable device; and
identifying a source of the snoring sound.

9. The method of claim 8, wherein identifying the source of the snoring sound further comprising:
determining the acoustic signal communicates via the SSM of the wearable device to identify a user wearing the wearable device.

10. The method of claim 1, further comprising:
transmitting a radio frequency (“RF”) signal including the acoustic signal and indication data representing the presence of the snoring condition to cause generation of noise cancellation signals based on the acoustic signal.

11. The method of claim 1, further comprising:
communicating a radio frequency (“RF”) signal to establish a wireless communication path with another wearable device and/or a media device.

12. An apparatus comprising:
a wearable housing;
a transducer disposed in the wearable housing and configured to receive acoustic energy of a snoring sound;
a snore detector configured to characterize the acoustic energy as being indicative of a presence of a snoring condition;
a snore manager configured to generate a notification signal to cause notification of the detection of the snoring sound; and.
a vibration generator configured to generate vibratory energy, responsive to the notification signal, to emit vibrations from the wearable housing,
wherein generation of the vibratory energy is indicative of the snoring condition.

13. The apparatus of claim 12, further comprising:
a skin surface microphone (“SSM”).

14. The apparatus of claim 12, further comprising:
a motion sensor configured to sense a level of motion; and
a motion analyzer configured to indicate that the level of motion is associated with the snoring condition.

15. The apparatus of claim 12, further comprising:
a memory configured to store data representing user characteristics; and
a user characterizer configured to determine the user characteristics indicate the acoustic energy is associated with the snoring condition,
wherein the snore detector is configured to generate a snore indicator signal including data representing the presence of the snoring condition.

16. The apparatus of claim 12, further comprising:
a radio frequency (“RF”) transmitter,
wherein the snore manager is configured to cause transmission via the RF transmitter an RF signal configured to initiate generation of one or more noise cancellation signals to form a null at a listening position other than a location that includes the wearable device.

17. A method comprising:
receiving an acoustic signal;
characterizing at a media device the acoustic signal as a snoring sound to determine presence of a snoring condition;
identifying a source of the snoring sound associated with a wearable device; and
transmitting a notification signal to cause a notification source to generate a notification of the detection of the snoring sound.

18. The method of claim 17, wherein receiving the acoustic signal comprises:
receiving the acoustic signal into an array of transducers in a first mode.

19. The method of claim 18, further comprising:
receiving different amplitudes of the acoustic signal into each of the transducers; and
determining a first location including with a user with the snoring condition.

20. The method of claim 18, further comprising:
transmitting noise cancellation signals via the array of transducers in a second mode to a second location at which to reduce or cancel an amplitude of the acoustic signal.
